(19) United States
(12) Patent Application Publication
(10) Pub. No.: US 2008/0108922 A1
(43) Pub. Date: May 8, 2008

(54) Joint brace with improved adjustable limb extension regulator

(75) Inventors: David Castillo, Aliso Viejo, CA (US); James Castillo, Los Almos, CA (US)

Correspondence Address: STETINA BRUNDA GARRED & BRUCKER, 75 ENTERPRISE, SUITE 250, ALISO VIEJO, CA, 92656, US

(73) Assignee: Asterisk.Asterisk, LLC

(21) Appl. No.: 11/591,899

(22) Filed: Nov. 2, 2006

(51) Int. Cl. A61F 5/00

(52) U.S. Cl. 602/16; 602/5

(57) ABSTRACT

There is provided a brace for stabilizing a uniting pivoting joint disposed between first and second limb structures of a living being. The brace comprises upper and lower frame members, each encompassing the first and second limb structures respectively, and a pivoting joint member comprised of two opposing pivoting assemblies. The assemblies include a limb extension slot having an adjustable length. The slot is located on at least one of the upper or lower frame members. The assemblies also include an exteriorly accessible limb extension regulator for regulating the limb extension slot length. The limb extension regulator is coupled to the limb extension slot. The pivoting assembly further includes an arm member comprised of upper and lower ends, each being pivotally connected to the upper and lower frame members, respectively. The arm member further includes a regulation portion retained in the limb extension slot.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

1. Field of Invention

This invention relates in general to braces for joint support. Specifically, the invention relates to an exteriorly positionable anatomical brace for stabilizing a uniting pivoting joint disposed between a first limb structure and a second limb structure. The brace includes an adjustable limb extension regulator to mitigate the chance of injury to the joint.

2. Description

The human body is comprised of a many different joints. Both injury and disease can affect the health, well-being, and operability of the various joints of the human body. Chief among such joints are the knee and elbow, where disease such as osteo-arthritis can curtail normal activity or where an injury such as a sports-related abuse or impact can prevent or severely limit continued activity. Many joint injuries occur when the ligaments holding the joint together tear, either completely or partially.

The knee joint is one of the body's most delicate joints making it prone to injury. The femur, tibia, and fibula are the bones within the leg that comprise the knee joint. The bones are connected by four ligaments. Two collateral ligaments are located on the outside of the knee and control the lateral motion of the knee. The anterior cruciate ligament (ACL) and posterior cruciate ligament (PCL) control rotation of the knee as well as the forwards and backwards motion of the knee. An injury to any joint, including the knee, is likely to occur when an excessive force is applied to the joint. The excessive force causes the bones to displace beyond its allowable limits, thereby causing the ligaments to tear.

One method of preventing or reducing the risk of joint injuries is to fit the joint with a brace. The brace restricts the joint's movement by providing added stability and support to the particular joint. Typically, a pivotal support member is placed adjacent to the particular joint, and support cuffs are attached to limb structures surrounding the joint to hold the brace in place. The joint pivots in a pivotal extension plane and provides stability to the joint.

Although a brace may provide added stability to a joint, the joint is still susceptible to hyper-extension in the pivotal extension plane. Many times, it is desirable to precisely limit or regulate the amount of brace extension within the pivotal extension plane.

To that end, several attempts have been made in the prior art to create a device that would regulate the distance of the pivotal extension plane of the joint. Such devices include braces with limb extension regulators which attempt to limit the pivotal extension plane of the brace. Although many braces are fitted with limb extension regulators that provide some degree of extension control, none of the limb extension regulators form an absolute rigid stop to prevent further extension.

As such, there is a need in the art for a joint brace with an improved limb extension regulator.

### BRIEF SUMMARY

According to an aspect of the present invention, there is provided an exteriorly positionable anatomical brace for stabilizing a uniting pivoting joint disposed between a first limb structure and a second limb structure of a living being. The brace comprises an upper frame member and lower frame member, each encompassing the first and second limb structures respectively. The brace further includes a pivoting joint member comprised of two opposing pivoting assemblies. Each assembly is respectively positionable on one side of the uniting pivoting joint. The pivoting assemblies include a limb extension slot having an adjustable length. The limb extension slot is located on at least one of the upper or lower frame members. The pivoting assemblies also include an exteriorly accessible limb extension regulator for regulating the limb extension slot length. The limb extension regulator is coupled to the limb extension slot. The pivoting assembly further includes an arm member. The arm member is comprised of an upper end and a lower end, each being pivotally connected to the upper frame member and lower frame member, respectively. The arm member further includes a regulation portion retained in the limb extension slot.

As earlier noted, proper joint care many times requires limited or regulated limb extension, with such control emanating at the pivoting joint member. Prior art controls have included a cable, which is inherently elastic. The elasticity in the cable may allow a brace to extend beyond the desired regulation point. If the brace extends beyond the desired regulation point, the ligaments may hyperextend or tear, causing injury. The limb extension regulator of the present invention provides an improved limb extension regulator. Specifically, according to various embodiments of the present invention, the limb extension regulator forms an absolute rigid stop to prevent the brace from extending beyond the desired regulation point. As such, a user may control the brace extension to prevent hyperextension or tearing of the joint ligaments.

According to other embodiments, the brace may include a screw threadably-coupled to the limb extension regulator. The screw may be tool-accessible for rotation and resulting lengthening or shortening of the limb extension slot length. As the screw rotates, the limb extension regulator moves along the shaft of the screw thereby lengthening or shortening the length of the limb extension slot.

In another embodiment of the invention, the brace may include an externally visible measurement scale for the length of the limb extension slot length. An externally visible measurement scale would allow a user to ensure that the length of the limb extension slot of each opposing pivoting assembly of the pivoting joint member can be made equal.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective lateral view of a knee brace with upper and lower frame members in place on a patient leg shown in phantom;

FIG. 2 is a perspective medial view of the knee brace of FIG. 1;

FIG. 3 is a medial perspective view of the upper frame member, including an externally visible measurement scale;

FIG. 4A is an outer side view of a pivoting joint assembly;

FIG. 4B is an inner side view of the pivoting joint assembly with the upper and lower caps shown detached from the upper and lower frame members, respectively;

FIG. 5A is an inner side view of the pivoting joint assembly, the assembly being the opposing assembly to that shown in FIGS. 4A and 4B;

FIG. 5B is an inner perspective view of the pivoting joint assembly, the assembly being the opposing assembly to that shown in FIGS. 4A and 4B; and

FIG. 6 is an exploded perspective view of the joint assembly.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

The ligaments holding a joint together are capable of withstanding a minimal level of tension. If a joint hyper-extends, the ligaments within that joint may fatigue and/or tear thereby causing injury to the joint. The present invention is an improved limb extension regulator to regulate the distance of the pivotal extension plane of a joint.

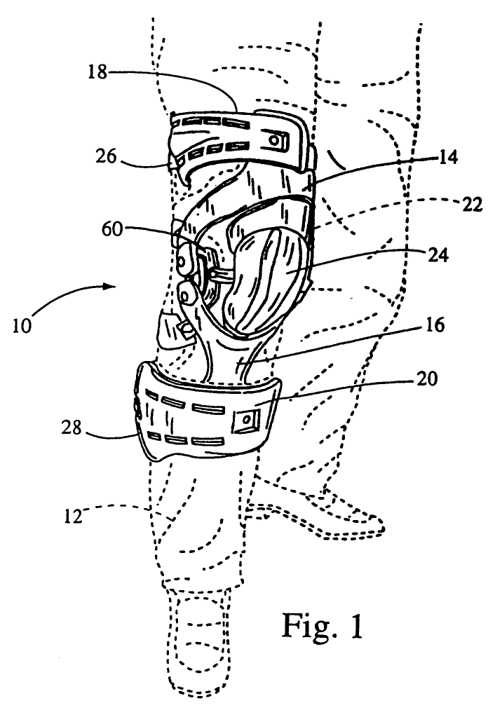

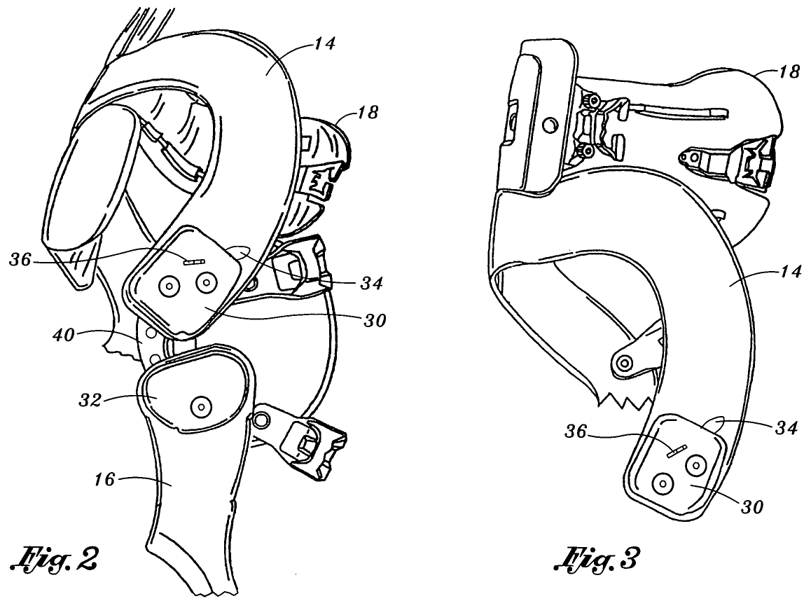

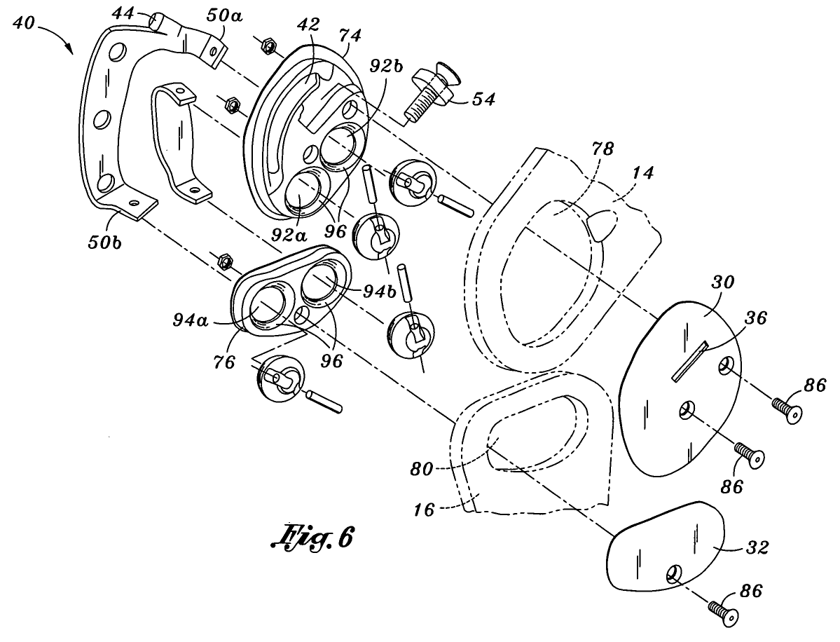

According to an aspect of the present invention, there is provided an exteriorly positionable anatomical brace for stabilizing a uniting pivoting joint disposed between a first limb structure and a second limb structure of a living being. Referring to FIG. 1-6, a knee brace 10 is shown in place on a leg 12 of a human being. Although FIG. 1 shows a knee brace 10, it is understood that the scope of the present invention shall include all anatomical braces for stabilizing a pivoting joint. This includes, but is not limited to, knee braces, elbow braces, and shoulder braces. The operation of the brace 10 is more fully described in U.S. Pat. No. 6,962,571, the disclosure of which is incorporated by reference.

The knee brace 10 comprises an upper frame member 14 and lower frame member 16, each encompassing the limb structures above and below the knee joint 22. As shown in FIG. 1, the limb structures are the upper and lower leg 12 of a human being. However, according to an aspect of the present invention, the brace 10 may be an elbow brace, in which case the upper and lower limb structures would be the forearm and bicep of a user.

The brace 10 further includes a pivoting joint member comprised of two opposing pivoting assemblies 50. The upper and lower frame members 14, 16 are united by the pivoting joint member. The pivoting joint member is pivotally connected to the upper and lower frame members 14, 16, thereby allowing the brace 10 to pivot, or flex within a pivot plane. Each pivoting assembly 50 is respectively positionable on one side of the uniting pivoting joint. As shown in brace 10, an assembly 50 is positioned on the medial side of the knee joint 22, and the other assembly 50 is positioned on the lateral side of the knee joint 22.

Referring now to FIG. 6, the assembly 50 includes an upper housing 74 and a lower housing 76 that fit, respectively into a complementarily shaped opening 78 of the upper frame member 14 and a complementarily shaped opening 80 of the lower frame member 16. Once so positioned, respective caps 30, 32 are held in place with conventional set screws 86. Those skilled in the art however will recognize that the housings 74 and 76 can be formed unitarily with the frame members 14 and 16. A lateral condyle 60 resides between the assembly 72 and the knee joint 22, as shown in FIG. 1. Both the upper and lower housings 74, 76 have two respective openings 92a, 92b and 94a, 94b each having respective sidewalls 96 shaped to nest a spherical shape. Disposed between two openings 92b, 94a of the housings 74, 76 is a forward arm member 40 having generally perpendicularly angled first and second ends 52a, 52b directable toward the openings 92b, 94a.

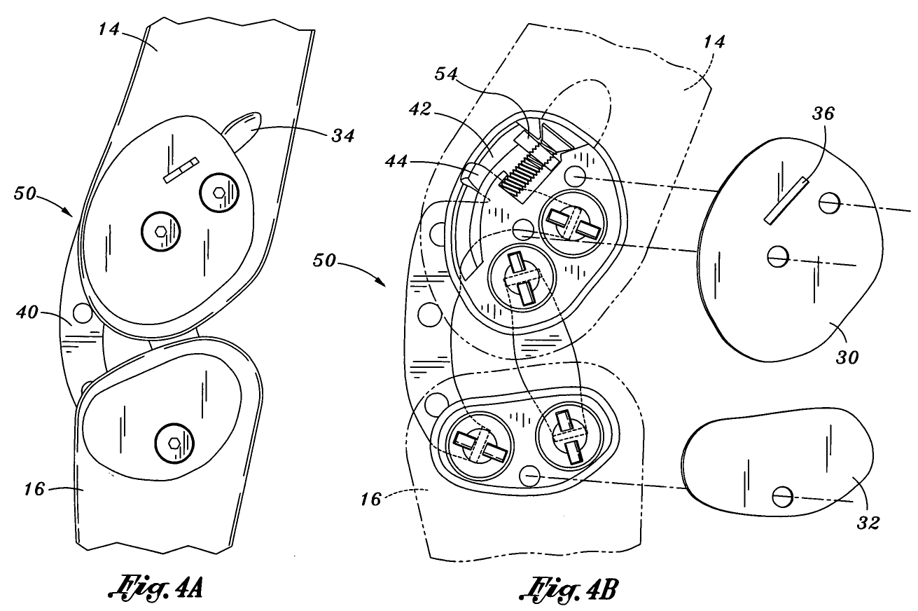

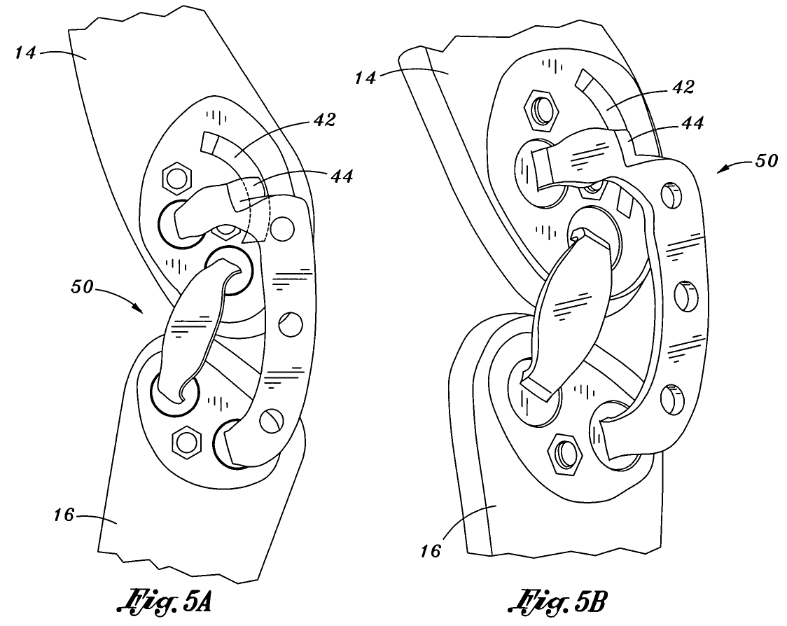

The assembly 50 also includes a limb extension slot 34 having an adjustable length. The limb extension slot 34 is located on at least one of the upper or lower frame members 14, 16. As shown in FIG. 4, the limb extension slot 34 is located in the upper frame member 14, however, it is understood that the slot 34 may alternatively be located in the lower frame member 16. The length of the limb extension slot 34 is regulated by an exteriorly accessible limb extension regulator 54, which will be discussed in more detail below.

The arm member 40 of the pivoting assembly 50 includes a regulation portion 44 which is retained in the limb extension slot 42. As the brace 10 pivots, the regulation portion 44 slides within the limb extension slot 42 until it reaches the limb extension regulator 54. The limb extension regulator 54 provides an absolute stop and prevents the regulation portion 44 of the arm member 40 from further sliding within the limb extension slot 42, thereby preventing any further extension of the brace 10. Thus the allowable extension of the brace 10 depends on the length of the limb extension slot 42.

The length of the limb extension slot 42 is adjustable. The limb extension regulator 54 controls the length of the limb extension slot 42. The limb extension regulator 54 may be exteriorly accessible via an access channel 34. The extension of the brace 10 is proportional to the length of the limb extension slot 42. For instance, as the length of the slot 42 increases, the allowable brace extension increase. Conversely, when the length of the slot 42 decreases, the allowable brace extension decreases.

According to an aspect of the present invention, the limb extension regulator 54 may be threadably-coupled to a screw, as shown in FIG. 4B and FIG. 6. The screw may be tool-accessible via access slot 34 for rotation allowing the screw to rotate within the limb extension slot 42. As the screw rotates, the limb extension regulator moves along the shaft of the screw. This results in lengthening or shortening of the limb extension slot length 42. As such, the user may control the length of the limb extension slot 42.

The limb extension regulator 54 of the present invention forms an absolute rigid stop. In other words, when the regulation portion of the arm member 40 abuts the limb extension regulator 54, the brace 10 can no longer pivot. There is no elasticity that would allow further pivoting, such as when a cable is used to regulate the extension of a brace. Therefore, a user may confidently control the allowable extension of brace 10, and know that the brace will not extend beyond the set distance.

According to an embodiment of the present invention, the brace 10 may include a measurement scale 36 to allow a user view the placement of the limb extension regulator 54. In one embodiment of the invention, the measurement scale 36 is externally visible to provide a means for allowing the user to view the position of the limb extension regulator 54 relative to the limb extension slot 42. The measurement scale 36 may be used to adjust the position of the limb extension regulator 54 on opposing pivoting assemblies 50 to ensure the length of the limb extension slot 42 of each opposing pivoting assembly 50 is made equal.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. An exteriorly positionable anatomical brace for stabilizing a uniting pivoting joint disposed between a first limb structure and a second limb structure of a living being, the brace comprising:
an upper frame member coupled to the first limb structure;
a lower frame member coupled to the second limb structure;
a pivoting joint member comprising two opposing pivoting assemblies each respectively positionable on one side of the uniting pivoting joint, each pivoting assembly including:
a limb extension slot having an adjustable length, the limb extension slot located on at least one of the upper or lower frame members;
a limb extension regulator for regulating the limb extension slot length, the limb extension regulator being coupled to the limb extension slot; and
an arm member comprising:
an upper end pivotally connected to the upper frame member;
a lower end pivotally connected to the lower frame member; and
a regulation portion retained in the limb extension slot.

2. An exteriorly positionable anatomical brace as claimed in claim 1, wherein the limb extension regulator is coupled to a threadably-engaged screw, the screw being tool-accessible for rotation and resulting lengthening or shortening of the limb extension slot length.

3. An exteriorly positionable anatomical brace as claimed in claim 1 additionally comprising an externally visible measurement scale allowing a user to adjust the limb extension regulator such that the length of the limb extension slot of each opposing pivoting assembly of the pivoting joint member can be made equal.

4. An exteriorly positionable anatomical brace as claimed in claim 1, wherein the limb extension slot is located on the upper frame member.
